(19) United States
(12) Patent Application Publication
(10) Pub. No.: US 2009/0291152 A1
(43) Pub. Date: Nov. 26, 2009

(54) DIBENZOTHIAZEPINE DERIVATIVES

(75) Inventors: Roger Tung, Lexington, MA (US); Scott Harbeson, Cambridge, MA (US)

Correspondence Address: EDWARDS ANGELL PALMER & DODGE LLP, P.O. BOX 55874, BOSTON, MA, 02205, US

(73) Assignee: Concert Pharmaceuticals Inc., Lexington, MA (US)

(21) Appl. No.: 12/425,957

(22) Filed: Apr. 17, 2009

Related U.S. Application Data
(63) Continuation of application No. PCT/US2007/022338, filed on Oct. 19, 2007.
(60) Provisional application No. 60/853,209, filed on Oct. 20, 2006.

(51) Int. Cl. A61K 31/554; C07D 417/04; A61K 33/00; A61P 25/18; A61P 25/24; A61P 25/16; A61P 25/28; C12N 5/00

(52) U.S. Cl. 424/722; 540/551; 514/211.13; 435/375

(57) ABSTRACT

This invention relates to novel 11-[4-[2-(2-Hydroxyethoxy)ethyl]piperazin-1-yl]dibenzo[b,f][1,4]thiazepine derivatives, their acceptable acid addition salts, solvates, hydrates and polymorphs thereof. The invention also provides compositions comprising a compound of this invention and the use of such compositions in methods of treating diseases and conditions beneficially treated by antagonists of seratonergic 5HT1A and 5HT2 receptors, dopaminergic D1 and D2 receptor, histaminergic H1 receptors, and/or adrenergic α1 and α2 receptors.

### RELATED APPLICATIONS

This application is a continuation of PCT Patent Application No. PCT/US2007/022338, filed Oct. 19, 2007, which claims the benefit of priority to U.S. provisional patent application No. 60/853,209, filed Oct. 20, 2006, the contents of which are incorporated herein by reference.

This invention relates to novel 11-[4-[2-(2-Hydroxyethoxy)ethyl]piperazin-1-yl]dibenzo[b,f][1,4]thiazepine derivatives, their acceptable acid addition salts, solvates, and hydrates thereof. The invention also provides compositions comprising a compound of this invention and the use of such compositions in methods of treating diseases and conditions beneficially treated by antagonists of seratonergic 5HT1A and 5HT2 receptors, dopaminergic D1 and D2 receptor, histaminergic H1 receptors, and/or andrenergic α1 and α2 receptors.

Quetiapine, chemically described as 11-[4-[2-(2-Hydroxyethoxy)ethyl]piperazin-1-yl]dibenzo[b,f][1,4]thiazepine has been shown to be an effective atypical antipsychotic agent useful as an antipsychotic or neuroleptic. It is an antagonist at multiple neurotransmitter receptors in the brain: e.g., seratonergic 5HT1A and 5HT2 dopaminergic; D1 and D2; histaminergic H1; and adrenergic α1 and α2. The interaction of quetiapine with H1 and adrenergic α1 receptors may explain the observed somnolence and orthostatic hypotension, respectively. Quetiapine may be used as an antipsychotic agent with a substantial reduction in the potential to cause side effects such as acute dystonia, acute dyskinesia, pseudo-Parkinsonism as well as tardive dyskinesia. See U.S. Pat. No. 4,879,288 and European Patent No. 0240228.

In clinical trials, quetiapine has been shown to be effective in the treatment of schizophrenia, mania, and bipolar disorder, and is approved for these indications. The compound is also in a number of clinical trials for the treatment of depression associated with bipolar disorders; agitation in Alzheimer's patients; alcoholism; generalized anxiety; major depression; borderline personality disorder; post-traumatic stress disorder; primary insomnia; dementia; and obsessive-compulsive disorder (see ClinicalTrials.gov web page).

Extensive metabolism of quetiapine occurs in the liver. Two major metabolites have been identified: the sulfoxide formed by CYP3A4 and the carboxylic acid formed by oxidation of the primary alcohol side-chain. Neither of these metabolites is active. A majority of the metabolites (73%) are excreted in the urine. Although renal excretion plays a significant role, no dosage adjustment is recommended for patients with severe renal impairment. However, patients with hepatic impairment have shown 3-fold increases in AUC and Cmax. Therefore, dosage adjustments may be needed in patients with hepatic impairment. See the label approved on Sep. 20, 2006 for NDA No. 020639, Drugs@FDA website; Grimm S W et al., Br J Clin Pharmacol 2006, 61, p. 58; Li K-Y et al., Methods Find Exp Clin Pharmacol 2005, 27, p. 83; Davis P C et al., J Pharm Biomed Anal 1999, 20, p. 271.

Quetiapine is currently administered as an immediate release formulation; however, a sustained release formulation (see WO 1997/045124) is currently in Phase III clinical trials and a NDA for use of the sustained release formulation in schizophrenia was submitted in the US on Jul. 18, 2006. Although dosing details for most of the trials are not available, in one trial (ClinicalTrials.gov web site; Identifier NCT00352469) the sustained release formulation is dosed up to 400 mg/day. A chemical modification of quetiapine that reduces the rates of metabolism and clearance may have significant therapeutic benefits, such as decreasing the dose and/or frequency of dosing.

Despite the beneficial activities of quetiapine, there is a continuing need for new compounds to treat the aforementioned diseases and conditions.

### DETAILED DESCRIPTION OF THE INVENTION

### Definitions

The terms “ameliorate” and “treat” are used interchangeably and both mean decrease, suppress, attenuate, diminish, arrest, or stabilize the development or progression of a disease (e.g., a psychotic disorder).

By “disease” is meant any condition or disorder that damages or interferes with the normal function of a cell, tissue, or organ.

Chemical naming terminology can be complex and different chemical names can often reasonably be applied to the same structure. To avoid any confusion, “Quetiapine” refers to a compound, wherein all hydrogen and all carbon atoms are present at their natural isotopic abundance percentages.

It will be recognized that some variation of natural isotopic abundance occurs in a synthesized compound depending upon the origin of chemical materials used in the synthesis. Thus, a preparation of quetiapine will inherently contain small amounts of deuterated and/or ¹³C-containing isotopologues. The concentration of naturally abundant stable hydrogen and carbon isotopes, notwithstanding this variation, is small and immaterial with respect to the degree of stable isotopic substitution of compounds of this invention. See for instance Wada E and Hanba Y, Seikagaku 1994 66: 15; Ganes L Z et al., Comp. Biochem. Physiol. A Mol. Integr. Physiol. 1998 119: 725. In a compound of this invention, when a particular position is designated as having deuterium, it is understood that the abundance of deuterium at that position is substantially greater than the natural abundance of deuterium, which is 0.015%. A position designated as having deuterium typically has a minimum isotopic enrichment factor of at least 3000 (45% deuterium incorporation) at each atom designated as deuterium in said compound.

The term “isotopic enrichment factor” as used herein means the ratio between the isotopic abundance and the natural abundance of a specified isotope.

In other embodiments, a compound of this invention has an isotopic enrichment factor for each designated deuterium atom of at least 3500 (52.5% deuterium incorporation at each designated deuterium atom), at least 4000 (60% deuterium incorporation), at least 4500 (67.5% deuterium incorporation), at least 5000 (75% deuterium incorporation), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation).

In the compounds of this invention any atom not specifically designated as a particular isotope is meant to represent any stable isotope of that atom. Unless otherwise stated, when a position is designated specifically as “H” or “hydrogen”, the position is understood to have hydrogen at its natural abundance isotopic composition.

In another embodiment, a “compound” of the invention contains less than 10%, preferably less than 6%, and more preferably less than 3% of all other isotopologues combined, including a form that lacks any deuterium or ¹³C. In certain aspects, the compound contains less than “X”% of all other isotopologues combined, including a form that lacks any deuterium or ¹³C; where X is any number between 0 and 10 (e.g., 1, 0.5, 0.001), inclusive. Compositions of matter that contain greater than 10% of all other isotopologues combined are referred to herein as “mixtures” and must meet the parameters set forth below. These limits of isotopic composition and all references to isotopic composition herein, refer solely to the relative amounts of deuterium/hydrogen and ¹³C/¹²C present in the active, free base form of the compound of Formula I, and do not include the isotopic composition of hydrolyzable portions of prodrugs, or of counterions.

The term “isotopologue” refers to species that differ from a specific compound of this invention only in the isotopic composition of their molecules or ions.

The term “compound” as used herein, is also intended to include salts, solvates or hydrates thereof.

A salt of a compound of this invention is formed between an acid and a basic group of the compound, such as an amino functional group, or a base and an acidic group of the compound, such as a carboxyl functional group. According to another preferred embodiment, the compound is a pharmaceutically acceptable acid addition salt.

The term “pharmaceutically acceptable,” as used herein, refers to a component that is, within the scope of sound medical judgment, suitable for use in contact with the tissues of humans and other mammals without undue toxicity, irritation, allergic response and the like, and are commensurate with a reasonable benefit/risk ratio. A “pharmaceutically acceptable salt” means any non-toxic salt that, upon administration to a recipient, is capable of providing, either directly or indirectly, a compound or a prodrug of a compound of this invention. A “pharmaceutically acceptable counterion” is an ionic portion of a salt that is not toxic when released from the salt upon administration to a recipient.

Acids commonly employed to form pharmaceutically acceptable salts include inorganic acids such as hydrogen bisulfide, hydrochloric, hydrobromic, hydroiodic, sulfuric and phosphoric acid, as well as organic acids such as para-toluenesulfonic, salicylic, tartaric, bitartaric, ascorbic, maleic, besylic, fumaric, gluconic, glucuronic, formic, glutamic, methanesulfonic, ethanesulfonic, benzenesulfonic, lactic, oxalic, para-bromophenylsulfonic, carbonic, succinic, citric, benzoic and acetic acid, and related inorganic and organic acids. Such pharmaceutically acceptable salts thus include sulfate, pyrosulfate, bisulfate, sulfite, bisulfite, phosphate, monohydrogenphosphate, dihydrogenphosphate, metaphosphate, pyrophosphate, chloride, bromide, iodide, acetate, propionate, decanoate, caprylate, acrylate, formate, isobutyrate, caprate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, dinitrobenzoate, hydroxybenzoate, methoxybenzoate, phthalate, terephathalate, sulfonate, xylenesulfonate, phenylacetate, phenylpropionate, phenylbutyrate, citrate, lactate, β-hydroxybutyrate,glycolate, maleate, tartrate, methanesulfonate, propanesulfonate, naphthalene-1-sulfonate, naphthalene-2-sulfonate, mandelate and the like salts. Preferred pharmaceutically acceptable acid addition salts include those formed with mineral acids such as hydrochloric acid and hydrobromic acid, and especially those formed with organic acids such as maleic acid.

As used herein, the term “hydrate” means a compound which further includes a stoichiometric or non-stoichiometric amount of water bound by non-covalent intermolecular forces.

As used herein, the term “solvate” means a compound which further includes a stoichiometric or non-stoichiometric amount of solvent such as water, acetone, ethanol, methanol, dichloromethane, 2-propanol, or the like, bound by non-covalent intermolecular forces.

The compounds of the present invention (e.g., compounds of Formula I), may contain an asymmetric carbon atom, for example, as the result of deuterium substitution or otherwise. As such, compounds of this invention can exist as either individual enantiomers, or mixtures of the two enantiomers. Accordingly, a compound of the present invention will include both racemic mixtures, and also individual respective stereoisomers that are substantially free from another possible stereoisomer. The term “substantially free of other stereoisomers” as used herein means less than 25% of other stereoisomers, preferably less than 10% of other stereoisomers, more preferably less than 5% of other stereoisomers and most preferably less than 2% of other stereoisomers, or less than “X”% of other stereoisomers (wherein X is a number between 0 and 100, inclusive) are present. Methods of obtaining or synthesizing an individual enantiomer for a given compound are well known in the art and may be applied as practicable to final compounds or to starting material or intermediates.

The term “stable compounds”, as used herein, refers to compounds which possess stability sufficient to allow manufacture and which maintain the integrity of the compound for a sufficient period of time to be useful for the purposes detailed herein (e.g., formulation into therapeutic products, intermediates for use in production of therapeutic compounds, isolatable or storable intermediate compounds, treating a disease or condition responsive to atypical antipsychotic agents).

A specific compound of this invention may also be referred to as a “heavy atom isotopic compound” to distinguish it from its lighter isotopologues when discussing mixtures of isotopologues.

The term “heavy atom” refers to isotopes of higher atomic weight than the predominant naturally occurring isotope.

Both “²H” and “D” refer to deuterium.

“Stereoisomer” refers to both enantiomers and diastereomers.

“tert” refers to tertiary.

“US” refers to the United States of America.

“FDA” refers to Food and Drug Administration.

“NDA” refers to New Drug Application.

The recitation of a listing of chemical groups in any definition of a variable herein includes definitions of that variable as any single group or combination of listed groups. The recitation of an embodiment for a variable herein includes that embodiment as any single embodiment or in combination with any other embodiments or portions thereof.

Throughout this specification, reference to “each Y” includes, independently, all “Y” groups (e.g., Y₁, Y₂, Y₃ and Y₄) where applicable. Reference to “each Z” includes, independently, all “Z” groups (e.g., Z₁, Z₂, Z₃ and Z₄) where applicable.

### Therapeutic Compounds

The present invention provides a compound of Formula I:

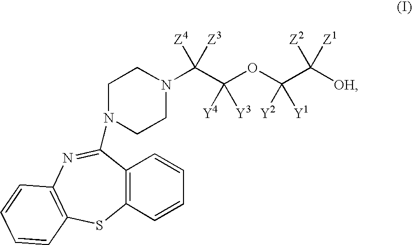

or a salt, hydrate, or solvate, or polymorph thereof; wherein:

each Z is independently selected from hydrogen or deuterium;

each Y is independently selected from hydrogen, deuterium or fluorine; and

at least one Z is deuterium.

In another embodiment, the compounds of this invention are those wherein Z and Z² are each deuterium.

In another embodiment, the compounds of this invention are those wherein Z³ and Z⁴ are each deuterium.

In one embodiment, the compounds of this invention are those wherein Z¹-Z⁴ are each deuterium.

In another embodiment, the compounds of this invention are those wherein Y¹ and Y² are each independently deuterium or fluorine.

In another embodiment, the compounds of this invention are those wherein Y¹ and Y² are each deuterium.

In another embodiment, the compounds of this invention are those wherein Y³ and Y⁴ are each independently deuterium or fluorine.

In another embodiment, the compounds of this invention are those wherein Y³ and Y⁴ are each deuterium.

In another embodiment, the compounds of this invention are those wherein Y¹-Y⁴ are each independently deuterium or fluorine.

In another embodiment, the compounds of this invention are those wherein Y¹-Y⁴ are each deuterium.

In another embodiment, the compounds of this invention are those wherein Y¹ and Y² are deuterium and Z¹ and Z² are deuterium.

In another embodiment, the compounds of this invention are those wherein Y¹ and Y² are fluorine and Z¹ and Z² are deuterium.

In another embodiment, the compounds of this invention are those wherein Y¹ and Y² are deuterium and Z¹-Z⁴ are deuterium.

In another embodiment, the compounds of this invention are those wherein Y¹ and Y² are fluorine and Z¹-Z⁴ are deuterium.

In another embodiment, the compounds of this invention are those wherein Y³ and Y⁴ are deuterium and Z³ and Z⁴ are deuterium.

In still another embodiment, the compound of formula I is selected from

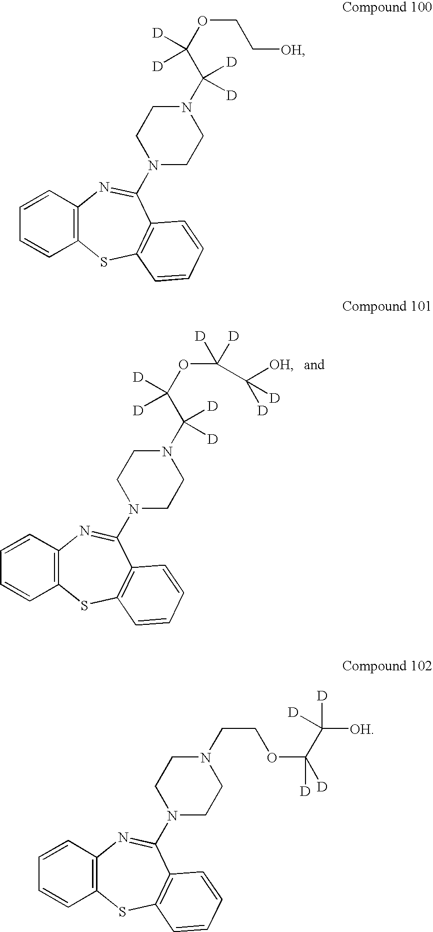

In still another set of embodiments, any atom not designated as deuterium in any of the embodiments set forth above is present at its natural isotopic abundance.

The synthesis of compounds of formula I can be readily achieved by synthetic chemists of ordinary skill. Relevant procedures and intermediates are disclosed, for instance, in U.S. Pat. No. 4,879,288, International Publication Nos. WO 05/014590, WO 05/028459, WO 05/028458, WO 05/028457, and WO 05/012274.

Such methods can be carried out utilizing corresponding deuterated and optionally, other isotope-containing reagents and/or intermediates to synthesize the compounds delineated herein, or invoking standard synthetic protocols known in the art for introducing isotopic atoms to a chemical structure. Certain intermediates can be used with or without purification (e.g., filtration, distillation, sublimation, crystallization, trituration, solid phase extraction, and chromatography).

### Exemplary Synthesis

A convenient method for producing compounds of formula I involves substitution of the amine hydrogen of 11-Piperazin-1-yl-dibenzo[b,f][1,4]thiazepine with an isotopically substituted 2-(2-Chloro-ethoxy)-ethanol under basic conditions (Scheme 1).

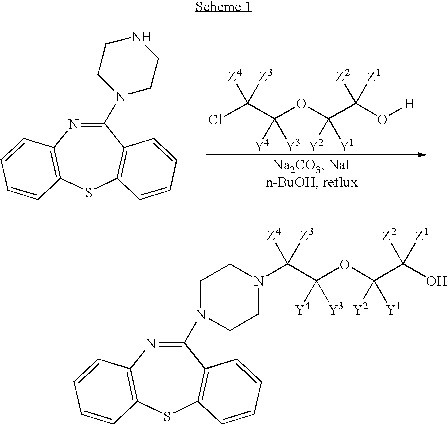

Another method for producing compounds of formula I is shown in Scheme 2.

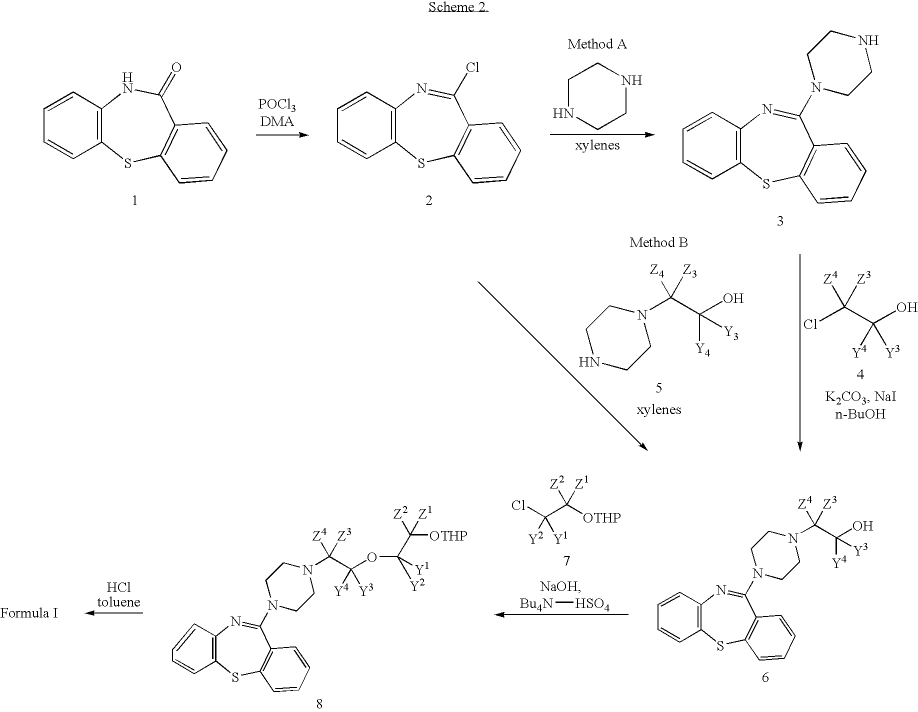

In Scheme 2, commercially-available dibenzo[b,f][1,4]thiazepin-11(10H)-one (1) is converted to (E)-11-chlorodibenzo[b,f][1,4]thiazepine 2 by treatment with POCl₃ and N,N-dimethylaniline. The chloride compound 2 is then treated with piperazine in xylenes to form (E)-11-(piperazin-1-yl)dibenzo[b,f][1,4]thiazepine 3, which is then treated with an appropriately deuterated chloroethanol in potassium carbonate, sodium iodide and n-butanol to afford alcohol 6. Alternatively, chloride compound 2 is treated with an appropriately deuterated hydroxyethyl piperazine 5 in xylenes to afford alcohol 6. Alcohol 6 is then treated with an appropriately deuterated alkylating agent 7 to afford the tetrahydropyran (THP)-protected intermediate 8, which is then deprotected with HCl in toluene to afford a compound of Formula I.

Other approaches to synthesizing compounds of formula I can readily be adapted from references cited herein. Variations of these procedures and their optimization are within the skill of the ordinary practitioner.

The specific approaches and compounds shown above are not intended to be limiting. Additional methods of synthesizing compounds of formula I and their synthetic precursors, including those within routes not explicitly shown in Schemes herein, are within the means of chemists of ordinary skill in the art. Methods for optimizing reaction conditions, if necessary minimizing competing by-products, are known in the art. Reaction optimization and scale-up may advantageously utilize high-speed parallel synthesis equipment and computer-controlled microreactors (e.g. Design And Optimization in Organic Synthesis, 2^{nd} Edition, Carlson R, Ed, 2005; Elsevier Science Ltd.; Jähnisch, K et al, Angew Chem Int Ed Engl 2004, 43: 406; and references therein).

In addition to the synthetic references cited herein, reaction schemes and protocols may be determined by the skilled artisan by use of commercially available structure-searchable database software, for instance, SciFinder® (CAS division of the American Chemical Society), STN® (CAS division of the American Chemical Society), CrossFire Beilstein® (Elsevier MDL), or internet search engines such as Google® or keyword databases such as the US Patent and Trademark Office text database.

The methods described herein may also additionally include steps, either before or after the steps described specifically herein, to add or remove suitable protecting groups in order to ultimately allow synthesis of the compounds herein. In addition, various synthetic steps may be performed in an alternate sequence or order to give the desired compounds. Synthetic chemistry transformations and protecting group methodologies (protection and deprotection) useful in synthesizing the applicable compounds are known in the art and include, for example, those described in R. Larock, Comprehensive Organic Transformations, VCH Publishers (1989); T. W. Greene and P. G. M. Wuts, Protective Groups in Organic Synthesis, 3 Ed., John Wiley and Sons (1999); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995) and subsequent editions thereof.

Combinations of substituents and variables envisioned by this invention are only those that result in the formation of stable compounds.

The invention further provides a mixture of a compound of this invention and its lighter isotopologues. These mixtures may occur, for instance, simply as the result of an inefficiency of incorporating the isotope at a given position; intentional or inadvertent exchange of protons for deuterium, e.g. exchange of bulk solvent for heteroatom-attached deuterium; or intentional mixtures of pure compounds.

### Compositions

The invention also provides compositions, especially pyrogen-free compositions, comprising an effective amount of a compound of Formula I (e.g., including any of the formulae herein), or a pharmaceutically acceptable salt, solvate, hydrate, polymorph or prodrug, if applicable, of said compound; and an acceptable carrier. Preferably, a composition of this invention is formulated for pharmaceutical use (“a pharmaceutical composition”), wherein the carrier is a pharmaceutically acceptable carrier. The carrier(s) must be “acceptable” in the sense of being compatible with the other ingredients of the formulation and, in the case of a pharmaceutically acceptable carrier, not deleterious to the recipient thereof in amounts typically used in medicaments.

Pharmaceutically acceptable carriers, adjuvants and vehicles that may be used in the pharmaceutical compositions of this invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

If required, the solubility and bioavailability of the compounds of the present invention in pharmaceutical compositions may be enhanced by methods well-known in the art. One method includes the use of lipid excipients in the formulation. See “Oral Lipid-Based Formulations: Enhancing the Bioavailability of Poorly Water-Soluble Drugs (Drugs and the Pharmaceutical Sciences),” David J. Hauss, ed. Informa Healthcare, 2007; and “Role of Lipid Excipients in Modifying Oral and Parenteral Drug Delivery: Basic Principles and Biological Examples,” Kishor M. Wasan, ed. Wiley-Interscience, 2006.

Another known method of enhancing bioavailability is the use of an amorphous form of a compound of this invention optionally formulated with a poloxamer, such as LUTROL™ and PLURONIC™ (BASF Corporation), or block copolymers of ethylene oxide and propylene oxide. See U.S. Pat. No. 7,014,866; and United States patent publications 20060094744 and 20060079502.

The pharmaceutical compositions of the invention include those suitable for oral, rectal, nasal, topical (including buccal and sublingual), vaginal or parenteral (including subcutaneous, intramuscular, intravenous and intradermal) administration. In certain embodiments, the compound of the formulae herein is administered transdermally (e.g., using a transdermal patch or iontophoretic techniques). Other formulations may conveniently be presented in unit dosage form, e.g., tablets and sustained release capsules, and in liposomes, and may be prepared by any methods well known in the art of pharmacy. See, for example, Remington's Pharmaceutical Sciences, Mack Publishing Company, Philadelphia, Pa. (17th ed. 1985).

Such preparative methods include the step of bringing into association with the molecule to be administered ingredients such as the carrier that constitutes one or more accessory ingredients. In general, the compositions are prepared by uniformly and intimately bringing into association the active ingredients with liquid carriers, liposomes or finely divided solid carriers or both, and then if necessary shaping the product.

In certain preferred embodiments, the compound is administered orally. Compositions of the present invention suitable for oral administration may be presented as discrete units such as capsules, sachets or tablets each containing a predetermined amount of the active ingredient; as a powder or granules; as a solution or a suspension in an aqueous liquid or a non-aqueous liquid; or as an oil-in-water liquid emulsion or a water-in-oil liquid emulsion, or packed in liposomes and as a bolus, etc. Soft gelatin capsules can be useful for containing such suspensions, which may beneficially increase the rate of compound absorption.

In the case of tablets for oral use, carriers that are commonly used include lactose and corn starch. Lubricating agents, such as magnesium stearate, are also typically added. For oral administration in a capsule form, useful diluents include lactose and dried cornstarch. When aqueous suspensions are administered orally, the active ingredient is combined with emulsifying and suspending agents. If desired, certain sweetening and/or flavoring and/or coloring agents may be added.

Compositions suitable for oral administration include lozenges comprising the ingredients in a flavored basis, usually sucrose and acacia or tragacanth; and pastilles comprising the active ingredient in an inert basis such as gelatin and glycerin, or sucrose and acacia.

Compositions suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example, sealed ampules and vials, and may be stored in a freeze dried (lyophilized) condition requiring only the addition of the sterile liquid carrier, for example water for injections, immediately prior to use. Extemporaneous injection solutions and suspensions may be prepared from sterile powders, granules and tablets.

Such injection solutions may be in the form, for example, of a sterile injectable aqueous or oleaginous suspension. This suspension may be formulated according to techniques known in the art using suitable dispersing or wetting agents (such as, for example, Tween 80) and suspending agents. The sterile injectable preparation may also be a sterile injectable solution or suspension in a non-toxic parenterally-acceptable diluent or solvent, for example, as a solution in 1,3-butanediol. Among the acceptable vehicles and solvents that may be employed are mannitol, water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant.

The pharmaceutical compositions of this invention may be administered in the form of suppositories for rectal administration. These compositions can be prepared by mixing a compound of this invention with a suitable non-irritating excipient which is solid at room temperature but liquid at the rectal temperature and therefore will melt in the rectum to release the active components. Such materials include, but are not limited to, cocoa butter, beeswax and polyethylene glycols.

The pharmaceutical compositions of this invention may be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art. Such administration is known to be effective with erectile dysfunction drugs: Rabinowitz J D and Zaffaroni A C, U.S. Pat. No. 6,803,031, assigned to Alexza Molecular Delivery Corporation.

Topical administration of the pharmaceutical compositions of this invention is especially useful when the desired treatment involves areas or organs readily accessible by topical application. For application topically to the skin, the pharmaceutical composition should be formulated with a suitable ointment containing the active components suspended or dissolved in a carrier. Carriers for topical administration of the compounds of this invention include, but are not limited to, mineral oil, liquid petroleum, white petroleum, propylene glycol, polyoxyethylene polyoxypropylene compound, emulsifying wax and water. Alternatively, the pharmaceutical composition can be formulated with a suitable lotion or cream containing the active compound suspended or dissolved in a carrier. Suitable carriers include, but are not limited to, mineral oil, sorbitan monostearate, polysorbate 60, cetyl esters wax, cetearyl alcohol, 2-octyldodecanol, benzyl alcohol and water. The pharmaceutical compositions of this invention may also be topically applied to the lower intestinal tract by rectal suppository formulation or in a suitable enema formulation. Topically-transdermal patches and iontophoretic administration are also included in this invention.

Other preferred dosage forms and formulations are those previously described for quetiapine in WO 06/081347; WO 05/070332; WO 01/021179; WO 97/045124, the disclosures of which are herein incorporated by reference.

Application of the subject therapeutics may be local, so as to be administered at the site of interest. Various techniques can be used for providing the subject compositions at the site of interest, such as injection, use of catheters, trocars, projectiles, pluronic gel, stents, sustained drug release polymers or other device which provides for internal access.

Thus, according to yet another embodiment, the compounds of this invention may be incorporated into compositions for coating an implantable medical device, such as prostheses, artificial valves, vascular grafts, stents, or catheters. Suitable coatings and the general preparation of coated implantable devices are known in the art and are exemplified in U.S. Pat. Nos. 6,099,562; 5,886,026; and 5,304,121. The coatings are typically biocompatible polymeric materials such as a hydrogel polymer, polymethyldisiloxane, polycaprolactone, polyethylene glycol, polylactic acid, ethylene vinyl acetate, and mixtures thereof. The coatings may optionally be further covered by a suitable topcoat of fluorosilicone, polysaccharides, polyethylene glycol, phospholipids or combinations thereof to impart controlled release characteristics in the composition. Coatings for invasive devices are to be included within the definition of pharmaceutically acceptable carrier, adjuvant or vehicle, as those terms are used herein.

According to another embodiment, the invention provides a method of coating an implantable medical device comprising the step of contacting said device with the coating composition described above. It will be obvious to those skilled in the art that the coating of the device will occur prior to implantation into a mammal.

According to another embodiment, the invention provides a method of impregnating an implantable drug release device comprising the step of contacting said drug release device with a compound or composition of this invention. Implantable drug release devices include, but are not limited to, biodegradable polymer capsules or bullets, non-degradable, diffusible polymer capsules and biodegradable polymer wafers.

According to another embodiment, the invention provides an implantable medical device coated with a compound or a composition comprising a compound of this invention, such that said compound is therapeutically active.

According to another embodiment, the invention provides an implantable drug release device impregnated with or containing a compound or a composition comprising a compound of this invention, such that said compound is released from said device and is therapeutically active.

Where an organ or tissue is accessible because of removal from the patient, such organ or tissue may be bathed in a medium containing a composition of this invention, a composition of this invention may be painted onto the organ, or a composition of this invention may be applied in any other convenient way.

In another embodiment, a composition of the present invention further comprises a second therapeutic agent. The second therapeutic agent includes any compound or therapeutic agent known to have or that demonstrates advantageous properties when administered with 11-[4-[2-(2-Hydroxyethoxy)ethyl]piperazin-1-yl]dibenzo[b,f][1,4]thiazepine. Such agents are described in detail in WO 06/081347; WO 05/070332; WO 01/021179; and WO 97/045124.

In another embodiment, the second therapeutic agent is an agent useful in the treatment or prevention of a disease or condition including, but not limited to, schizophrenia; schizoaffective disorders; mania (manic disorder); bipolar I disorder; bipolar II disorder; depression associated with bipolar disorders; unipolar depression; agitation and/or insomnia in Alzheimer's patients; agitation and/or psychosis in dementia and/or Parkinsonism; alcoholism; sleep disorders associated with alcohol abstention in alcoholics; substance-related disorders; generalized agitation; generalized anxiety; anxiety disorders; anxiety neuroses; major depression (major depressive disorder); borderline personality disorder; post-traumatic stress disorder; primary insomnia; dementia; anorexia nervosa; social phobia; manic-depressive psychoses; mood disorders; psychotic disorders; psychosis; and obsessive-compulsive disorder.

In a more specific embodiment, the second therapeutic agent co-formulated with a compound of this invention is an agent useful in the treatment of schizophrenia, mania, and bipolar disorder.

In another embodiment, the second therapeutic agent co-formulated with a compound of this invention is sabcomeline (see WO 06/067496); a nicotine acetylcholine alpha 7 receptor agonist as described in WO 06/048294; a reversible monoamine oxidase inhibitor, which is selected from moclobemide, brofaromine, befloxatone, and toloxatone; a selective serotonin reuptake inhibitor, which is selected from gluoxetine, citalopram, excitalopram, fluvoxamine, sertraline, and paroxetine (see WO 05/053703); a serotonin/norepinephrine reuptake inhibitor (SNRI); a dopamine D1 antagonist such as pergolide (JP 05/060286); zolmitriptan (WO 03/018009); an anticonvulsant agent, such as divalproex; lithium; or an anti-hypertensive agent, such as guanfacine.

In another embodiment, the invention provides separate dosage forms of a compound of this invention and a second therapeutic agent that are associated with one another. The term “associated with one another” as used herein means that the separate dosage forms are packaged together or otherwise attached to one another such that it is readily apparent that the separate dosage forms are intended to be sold and administered together (within less than 24 hours of one another, consecutively or simultaneously).

In the pharmaceutical compositions of the invention, the compound of the present invention is present in an effective amount. As used herein, the term “effective amount” refers to an amount which, when administered in a proper dosing regimen, is sufficient to reduce or ameliorate the severity, duration or progression of the disorder being treated, prevent the advancement of the disorder being treated, cause the regression of the disorder being treated, or enhance or improve the prophylactic or therapeutic effect(s) of another therapy.

The interrelationship of dosages for animals and humans (based on milligrams per meter squared of body surface) is described in Freireich et al., (1966) Cancer Chemother Rep 50: 219. Body surface area may be approximately determined from height and weight of the patient. See, e.g., Scientific Tables, Geigy Pharmaceuticals, Ardsley, N.Y., 1970, 537. An effective amount of a compound of this invention can range from about 1.0 mg/kg to about 200 mg/kg, more preferably 1.0 mg/kg to about 50 mg/kg, more preferably 0.1 mg/kg to about 40 mg/kg. Effective doses will also vary, as recognized by those skilled in the art, depending on the diseases treated, the severity of the disease, the route of administration, the sex, age and general health condition of the patient, excipient usage, the possibility of co-usage with other therapeutic treatments such as use of other agents and the judgment of the treating physician.

For pharmaceutical compositions that comprise a second therapeutic agent, an effective amount of the second therapeutic agent is between about 20% and 100% of the dosage normally utilized in a monotherapy regime using just that agent. Preferably, an effective amount is between about 70% and 100% of the normal monotherapeutic dose. The normal monotherapeutic dosages of these second therapeutic agents are well known in the art. See, e.g., Wells et al., eds., Pharmacotherapy Handbook, 2nd Edition, Appleton and Lange, Stamford, Conn. (2000); PDR Pharmacopoeia, Tarascon Pocket Pharmacopoeia 2000, Deluxe Edition, Tarascon Publishing, Loma Linda, Calif. (2000), each of which references are entirely incorporated herein by reference.

It is expected that some of the second therapeutic agents referenced above will act synergistically with the compounds of this invention. When this occurs, its will allow the effective dosage of the second therapeutic agent and/or the compound of this invention to be reduced from that required in a monotherapy. This has the advantage of minimizing toxic side effects of either the second therapeutic agent of a compound of this invention, synergistic improvements in efficacy, improved ease of administration or use and/or reduced overall expense of compound preparation or formulation.

### Methods of Treatment

In another embodiment, the invention provides a method of modulating the activity of one or more of seratonergic 5HT1A or 5HT2 receptors, dopaminergic D1 or D2 receptor, histaminergic H1 receptors, or adrenergic a1 or a2 receptors in a cell comprising contacting the cell with one or more compounds or compositions of this invention.

According to another embodiment, the invention provides a method of treating a subject suffering from or susceptible to a disease that is beneficially treated by quetiapine comprising the step of administering to said subject an effective amount of a compound or a composition of this invention. Such diseases are well known in the art and are disclosed in WO 06/081347; WO 05/070332; WO 01/021179; and WO 97/045124.

In a preferred embodiment, the method of this invention is used to treat a subject suffering from or susceptible to a disease or condition selected from schizophrenia; schizoaffective disorders; mania (manic disorder); bipolar I disorder; bipolar II disorder; depression associated with bipolar disorders; unipolar depression; agitation and/or insomnia in Alzheimer's patients; agitation and/or psychosis in dementia and/or Parkinsonism; alcoholism; sleep disorders associated with alcohol abstention in alcoholics; substance-related disorders; generalized agitation; generalized anxiety; anxiety disorders; anxiety neuroses; major depression (major depressive disorder); borderline personality disorder; post-traumatic stress disorder; primary insomnia; dementia; anorexia nervosa; social phobia; manic-depressive psychoses; mood disorders; psychotic disorders; psychosis; and obsessive-compulsive disorder. Methods delineated herein include those wherein the subject is identified as in need of a particular stated treatment. Identifying a subject in need of such treatment can be in the judgment of a subject or a health care professional and can be subjective (e.g. opinion) or objective (e.g. measurable by a test or diagnostic method).

In a more specific embodiment, the method of this invention is used to treat a patient suffering from or susceptible to bipolar I disorder, or schizophrenia.

In another embodiment, the above method of treatment comprises the further step of co-administering to the patient one or more second therapeutic agents. The choice of second therapeutic agent may be made from any second therapeutic agent known to be useful for co-administration with quetiapine. Such agents are described in PCT patent publications WO 06/081347; WO 05/070332; WO 01/021179; and WO 97/045124, as are the conditions and diseases for which each may be used in conjunction with a compound of this invention.

In another embodiment, the second therapeutic agent co-administered with a compound of this invention is sabcomeline (see WO 06/067496); a nicotine acetylcholine alpha 7 receptor agonist as described in WO 06/048294; a reversible monoamine oxidase inhibitor, which is selected from moclobemide, brofaromine, befloxatone, and toloxatone; a selective serotonin reuptake inhibitor (SSRI), which is selected from gluoxetine, citalopram, excitalopram, fluvoxamine, sertraline, and paroxetine (see WO 05/053703); a serotonin/norepinephrine reuptake inhibitor (SNRI); a dopamine D1 antagonist such as pergolide (JP 05/060286); zolmitriptan (WO 03/018009); an anticonvulsant agent, such as divalproex; lithium; and an anti-hypertensive agent, such as guanfacine.

In particular, the invention provides a method of treating a patient suffering from or susceptible to anxiety and anxiety related disorders comprising the step of co-administering to the patient a pharmaceutical composition comprising a compound of formula I; and a SSRI or a SNRI.

In another embodiment, the invention provides a method of treating a patient suffering from or susceptible to Alzheimer's disease or another form of dementia comprising the step of co-administering to the patient a pharmaceutical composition comprising a compound of formula I; and divalproex.

In still another embodiment, the invention provides a method of treating a patient suffering from or susceptible to schizophrenia or a schizophrenic disorder comprising the step of co-administering to the patient a pharmaceutical composition comprising a compound of formula I; and guanfacine.

In still another embodiment, the invention provides a method of treating a patient suffering from or susceptible to bipolar I disorder comprising the step of co-administering to the patient a pharmaceutical composition comprising a compound of formula I; and a second therapeutic agent selected from lithium and divaloprex.

The term “co-administered” as used herein means that the second therapeutic agent may be administered together with a compound of this invention as part of a single dosage form (such as a composition of this invention comprising a compound of the invention and an second therapeutic agent as described above) or as separate, multiple dosage forms. Alternatively, the additional agent may be administered prior to, consecutively with, or following the administration of a compound of this invention. In such combination therapy treatment, both the compounds of this invention and the second therapeutic agent(s) are administered by conventional methods. The administration of a composition of this invention comprising both a compound of the invention and a second therapeutic agent to a subject does not preclude the separate administration of that same therapeutic agent, any other second therapeutic agent or any compound of this invention to said subject at another time during a course of treatment.

Effective amounts of these second therapeutic agents are well known to those skilled in the art and guidance for dosing may be found in patents and published patent applications referenced herein, as well as in Wells et al., eds., Pharmacotherapy Handbook, 2nd Edition, Appleton and Lange, Stamford, Conn. (2000); PDR Pharmacopoeia, Tarascon Pocket Pharmacopoeia 2000, Deluxe Edition, Tarascon Publishing, Loma Linda, Calif. (2000), and other medical texts. However, it is well within the skilled artisan's purview to determine the second therapeutic agent's optimal effective-amount range.

In one embodiment of the invention where a second therapeutic agent is administered to a subject, the effective amount of the compound of this invention is less than its effective amount would be where the second therapeutic agent is not administered. In another embodiment, the effective amount of the second therapeutic agent is less than its effective amount would be where the compound of this invention is not administered. In this way, undesired side effects associated with high doses of either agent may be minimized. Other potential advantages (including without limitation improved dosing regimens and/or reduced drug cost) will be apparent to those of skill in the art.

In yet another aspect, the invention provides the use of a compound of formula I alone or together with one or more of the above-described second therapeutic agents in the manufacture of a medicament, either as a single composition or as separate dosage forms, for treatment or prevention in a subject of a disease, disorder or symptom set forth above. Another aspect of the invention is a compound of the formulae herein for use in the treatment or prevention in a subject of a disease, disorder or symptom thereof delineated herein.

### Diagnostic Methods and Kits

The compounds and compositions of this invention are also useful as reagents in methods for determining the concentration of quetiapine in solution or biological sample such as plasma, examining the metabolism of quetiapine and other analytical studies.

According to one embodiment, the invention provides a method of determining the concentration, in a solution or a biological sample, of quetiapine, comprising the steps of:
a) adding a known concentration of a compound of Formula I to the solution of biological sample;
b) subjecting the solution or biological sample to a measuring device that distinguishes quetiapine from a compound of Formula I;
c) calibrating the measuring device to correlate the detected quantity of the compound of Formula I with the known concentration of the compound of Formula I added to the biological sample or solution; and
d) measuring the quantity of quetiapine in the biological sample with said calibrated measuring device; and
e) determining the concentration of quetiapine in the solution of sample using the correlation between detected quantity and concentration obtained for a compound of Formula I.

Measuring devices that can distinguish quetiapine from the corresponding compound of Formula I include any measuring device that can distinguish between two compounds that differ from one another only in isotopic abundance. Exemplary measuring devices include a mass spectrometer, NMR spectrometer, or IR spectrometer.

In another embodiment, the invention provides a method of evaluating the metabolic stability of a compound of Formula I comprising the steps of contacting the compound of Formula I with a metabolizing enzyme source for a period of time and comparing the amount of the compound of Formula I with the metabolic products of the compound of Formula I after the period of time.

In a related embodiment, the invention provides a method of evaluating the metabolic stability of a compound of Formula I in a patient following administration of the compound of Formula I. This method comprises the steps of obtaining a serum, urine or feces sample from the patient at a period of time following the administration of the compound of Formula I to the subject; and comparing the amount of the compound of Formula I with the metabolic products of the compound of Formula I in the serum, urine or feces sample.

The present invention also provides kits for use to treat schizophrenia; mania; bipolar disorder; depression associated with bipolar disorders; agitation in Alzheimer's patients; alcoholism; generalized anxiety; major depression; borderline personality disorder; post-traumatic stress disorder; primary insomnia; dementia; and/or obsessive-compulsive disorder. These kits comprise: a) a pharmaceutical composition comprising a compound of Formula I or a salt thereof; or a prodrug, or a salt of a prodrug thereof; or a hydrate, solvate, or polymorph thereof, wherein said pharmaceutical composition is in a container; and b) instructions describing a method of using the pharmaceutical composition to treat depression associated with bipolar disorders; agitation in Alzheimer's patients; alcoholism; generalized anxiety; major depression; borderline personality disorder; post-traumatic stress disorder; primary insomnia; dementia; and/or obsessive-compulsive disorder.

The container may be any vessel or other sealed or sealable apparatus that can hold said pharmaceutical composition. Examples include bottles, divided or multi-chambered holders bottles, wherein each division or chamber comprises a single dose of said composition, a divided foil packet wherein each division comprises a single dose of said composition, or a dispenser that dispenses single doses of said composition. The container can be in any conventional shape or form as known in the art which is made of a pharmaceutically acceptable material, for example a paper or cardboard box, a glass or plastic bottle or jar, a re-sealable bag (for example, to hold a “refill” of tablets for placement into a different container), or a blister pack with individual doses for pressing out of the pack according to a therapeutic schedule. The container employed can depend on the exact dosage form involved, for example a conventional cardboard box would not generally be used to hold a liquid suspension. It is feasible that more than one container can be used together in a single package to market a single dosage form. For example, tablets may be contained in a bottle, which is in turn contained within a box. Preferably, the container is a blister pack.

The kit may additionally comprise a memory aid of the type containing information and/or instructions for the physician, pharmacist or subject. Such memory aids include numbers printed on each chamber or division containing a dosage that corresponds with the days of the regimen which the tablets or capsules so specified should be ingested, or days of the week printed on each chamber or division, or a card which contains the same type of information. For single dose dispensers, memory aids further include a mechanical counter which indicates the number of daily doses that have been dispensed and a battery-powered micro-chip memory coupled with a liquid crystal readout and/or audible reminder signal which, for example, reads out the date that the last daily dose has been taken and/or reminds one when the next dose is to be taken. Other memory aids useful in such kits are a calendar printed on a card, as well as other variations that will be readily apparent.

The kits of this invention may also comprise a device to administer or to measure out a unit dose of the pharmaceutical composition. Such device may include an inhaler if said composition is an inhalable composition; a syringe and needle if said composition is an injectable composition; a syringe, spoon, pump, or a vessel with or without volume markings if said composition is an oral liquid composition; or any other measuring or delivery device appropriate to the dosage formulation of the composition present in the kit.

In certain embodiment, the kits of this invention may comprise in a separate vessel of container a pharmaceutical composition comprising a second therapeutic agent, such as one of those listed above for use for co-administration with a compound of this invention.

### EXAMPLES

### Example 1

### Preparations of Compounds of Formula I (Methods A and B)

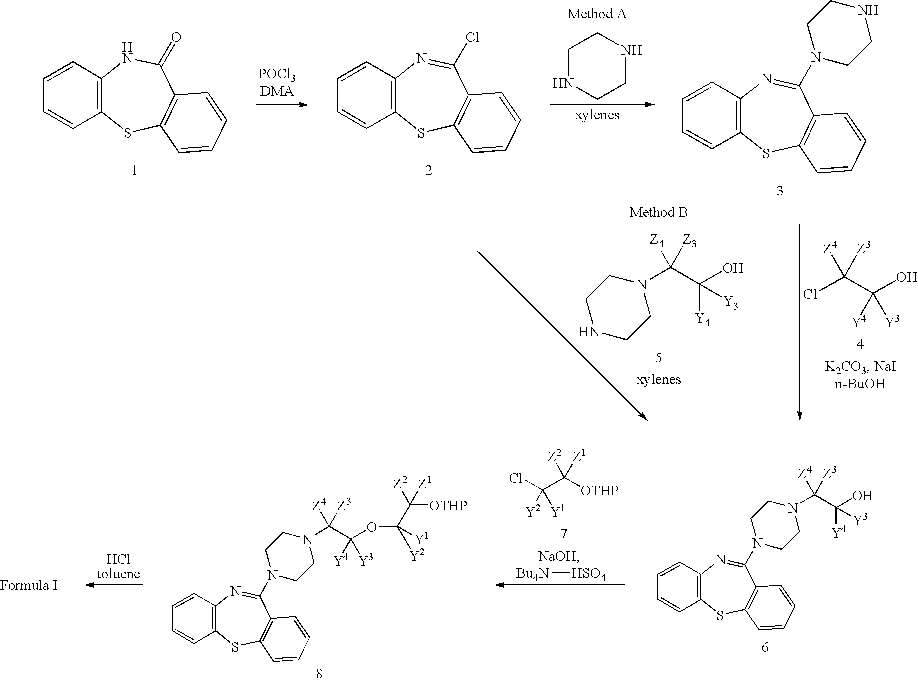

Preparation of (E)-11-chlorodibenzo[b,f][1,4]thiazepine (2). Commercially-available dibenzo[b,f][1,4]thiazepin-11(10H)-one (1; 1.50 g, 6.60 mmol) was suspended in POCl₃ (10 mL) in a round-bottom flask. To the thick, stirring slurry was added N,N-dimethylaniline (0.25 mL, 2.0 mmol). The reaction mixture was heated in a 130° C. oil bath under a condenser for 4 h. The resulting clear yellow solution was cooled to RT and concentrated on a rotary evaporator to yield a brown residue, which was dissolved in xylenes (40 mL) and poured into ice water (40 mL). The mixture was transferred to a separatory funnel and shaken, and the layers were separated. The organic layer was washed with 1N HCl, water, and brine. The organic layer was dried over magnesium sulfate, filtered and concentrated on a rotary evaporator to a reduced volume (approximately 10-20 mL.) This solution was carried on immediately to the next synthetic step.

Method A. Preparation of (E)-11-(piperazin-1-yl)dibenzo[b,f][1,4]thiazepine (3). Piperazine (796 mg, 9.24 mmol) was added to a stirring solution of 2 (6.60 mmol) in xylenes (approximately 10-20 mL). The reaction mixture was heated in a 170° C. oil bath under a condenser overnight. After 17 h the reaction was cooled to RT and diluted with Et₂O (50 mL) and water (50 mL). The cloudy mixture was transferred to a separatory funnel, shaken, and the layers were separated. The aqueous layer was extracted with Et₂O (3×40 mL), using brine to break any observed emulsions. The combined organic layers were washed with water (2×50 mL) and then extracted with 1N HCl (3×50 mL). The aqueous acidic layers were combined and made alkaline by addition of 5N NaOH until cloudy. The cloudy aqueous layer was quickly extracted with Et₂O (4×50 mL). The combined Et₂O extracts were concentrated immediately to afford 3 as a white solid residue, 1.685 g. This material was carried forward in crude form.

¹H NMR (CDCl₃) δ 2.87-2.93 (m, 2H), 2.98-3.01 (m, 2H), 3.42-3.68 (m, 4H), 6.86-6.92 (m, 1H), 7.05-7.11 (m, 1H), 7.15-7.21 (m, 1H), 7.27-7.41 (m, 4H), 7.49-7.54 (m, 1H). LCMS m/z 295.9 [M+H].

Preparation of Appropriately Deuterated (E)-11-[4-hydroxyethyl-(piperazin-1-yl)]dibenzo[b,f][1,4]thiazepine 6. A mixture of 3 (1.05 g, 3.55 mmol) in n-BuOH (9 mL) was sonicated briefly to afford a fine slurry. To the slurry was added K₂CO₃ (491 mg, 3.55 mmol), NaI (267 mg, 1.78 mmol) and the appropriately-deuterated chloroethanol 4 (4.26 mmol). The slurry was stirred in a 102° C. oil bath under a condenser for 24 h to 48 h. The reaction mixture was cooled and diluted with MeOH (120 mL) and stirred well for 5 min. The slurry was filtered through a medium fritted funnel and the filter cake was washed well with MeOH. The collected MeOH filtrate was concentrated on a rotary evaporator to afford a yellow solid which was dissolved in CH₂Cl₂ (200 mL) and transferred to a separatory funnel. The organic layer was washed with water and the layers were separated and retained. The organic layer was diluted with an equal volume of Et₂O until it clarified. The retained aqueous layer was extracted with Et₂O and the combined organic layers were washed with brine. The organic layer was dried over magnesium sulfate, filtered and concentrated to afford 6.

Method B. Preparation of Appropriately Deuterated (E)-2-(4-(dibenzo[b,f][1,4]thiazepin-11-yl)piperazin-1-yl)ethanol 6. The appropriately-deuterated amine 5 (9.24 mmol) was added to a stirring solution of 2 (6.60 mmol) in xylenes (approximately 10-20 mL). The reaction mixture was heated in a 170° C. oil bath under a condenser overnight. After 12 h to 18 h the reaction was cooled to RT and diluted with Et₂O (60 mL) and water (60 mL). The cloudy mixture was transferred to a separatory funnel, shaken, and the layers were separated. The aqueous layer was extracted with Et₂O (40 mL), using brine to break any observed emulsions. The combined organic layers were washed with water (2×50 mL) and then extracted with 1N HCl (3×50 mL). The aqueous acidic layers were combined and made alkaline by addition of 5N NaOH until cloudy. The cloudy aqueous layer was quickly extracted with Et₂O (4×50 mL). The combined Et₂O extracts were washed with brine, dried over magnesium sulfate, filtered and concentrated on a rotary evaporator to afford 6.

Preparation of (E)-2-(4-(dibenzo[b,f][1,4]thiazepin-11-yl)piperazin-1-yl)ethanol-d₄ 6a. (E)-2-(4-(dibenzo[b,f][1,4]thiazepin-11-yl)piperazin-1-yl)ethanol-d₄

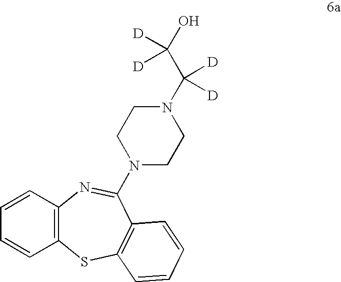

was prepared according to general method A in 52% yield. This material was carried forward in crude form.

¹H NMR (CDCl₃) δ 2.52-2.57 (m, 2H), 2.63-2.68 (m, 2H), 3.47-3.60 (m, 2H), 3.65 (t, 2H, J=6.4), 6.89 (t, 1H, J=7.6), 7.07 (d, 1H, J=7.6), 7.15-7.19 (m, 1H), 7.27-7.36 (m, 3H), 7.39 (d, 1H, J=7.6) 7.51 (d, 1H, J=7.3). LCMS m/z 343.9 [M+H].

Preparation of (E)-2-(4-(dibenzo[b,f][1,4]thiazepin-11-yl)piperazin-1-yl)ethanol 6b. (E)-2-(4-(dibenzo[b,f][1,4]thiazepin-11-yl)piperazin-1-yl)ethanol

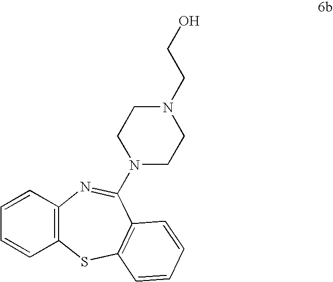

was prepared according to general method B in 53% yield over two steps from 1. This material was carried forward in crude form.

¹H NMR (CDCl₃) δ 2.52-2.57 (m, 2H), 2.59-2.65 (m, 2H), 2.60 (t, 2H, J=5.4), 3.45-3.61 (m, 4H), 3.65 (t, 2H, J=5.4), 6.89 (td, 1H, J=1.5, 7.6), 7.07 (dd, 1H, J=1.5, 7.9), 7.17 (td, 1H, J=1.5, 7.3), 7.27-7.36 (m, 3H), 7.39 (dd, 1H, J=1.5, 7.6), 7.50-7.52 (m, 1H). LCMS m/z 340.0 [M+H].

Preparation of Appropriately Deuterated (E)-11-(4-(2-(2-(tetrahydro-2H-pyran-2-yloxy)ethoxy)ethyl)piperazin-1-yl)dibenzo[b,f][1,4]thiazepine 8. A round-bottom flask was charged with 6 (0.786 mmol), 50% aq NaOH (1.6 mL), appropriately-deuterated alkylating agent 7 (3.54 mmol), and Bu₄N—HSO₄ (26.8 mg, 0.079 mmol). The mixture was stirred in a 60° C. oil bath for 24 h to 48 h. The reaction mixture was cooled to RT and poured into a separatory funnel containing Et₂O (30 mL) and water (30 mL). The layers were shaken and separated. The aqueous layer was extracted with Et₂O (2×15 mL) and the combined organic layers were washed with brine. The organic layer was dried over magnesium sulfate, filtered and concentrated on a rotary evaporator to afford 8.

Preparation of (E)-11-(4-(2-(2-(tetrahydro-2H-pyran-2-yloxy)ethoxy)ethyl-d₄)piperazin-1-yl)dibenzo[b,f][1,4]thiazepine 8a. (E)-11-(4-(2-(2-(tetrahydro-2H-pyran-2-yloxy)ethoxy)ethyl-d₄)piperazin-1-yl)dibenzo[b,f][1,4]thiazepine,

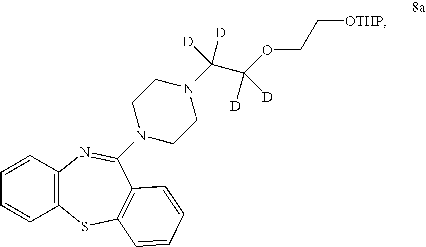

was prepared as described above. This material was purified via column chromatography (0%-10% MeOH/CH₂Cl₂) to afford 56% yield.

¹H NMR (CDCl₃) δ 1.48-1.85 (m, 6H), 2.52-2.66 (m, 4H), 3.47-3.66 (m, 7H), 3.83-3.89 (m, 3H), 4.63 (t, 1H, J=3.9), 6.87 (td, 1H, J=1.5, 7.6), 7.06 (dd, 1H, J=1.5, 7.9), 7.16 (td, 1H, J=1.5, 7.9), 7.28-7.34 (m, 3H), 7.38 (dd, 1H, J=1.5, 7.9), 7.49-7.51 (m, 1H). LCMS m/z 472.0 [M+H].

Preparation of (E)-11-(4-(2-(2-(tetrahydro-2H-pyran-2-yloxy)ethoxy-d₄)ethyl-d4)piperazin-1-yl)dibenzo[b,f][1,4]thiazepine 8b. (E)-11-(4-(2-(2-(tetrahydro-2H-pyran-2-yloxy)ethoxy-d4)ethyl-d4)piperazin-1-yl)dibenzo[b,f][1,4]thiazepine,

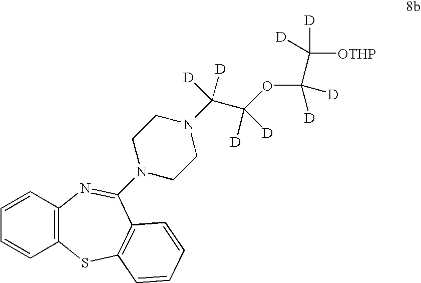

was prepared as described above. This material was purified via column chromatography (0%-5% MeOH/CH₂Cl₂) to afford 22% yield.

¹H NMR (CDCl₃) δ 1.47-1.84 (m, 6H), 2.52-2.57 (m, 2H), 2.61-2.70 (m, 2H), 3.47-3.66 (m, 5H), 3.84-3.89 (m, 1H), 4.62 (t, 1H, J=4.3), 6.87 (td, 1H, J=1.5, 7.6), 7.06 (dd, 1H, J=1.5, 7.9), 7.16 (td, 1H, J=1.5, 7.6), 7.28-7.34 (m, 3H), 7.38 (dd, 1H, J=1.5, 7.9), 7.49-7.51 (m, 1H). LCMS m/z 476.0 [M+H].

Preparation of (E)-11-(4-(2-(2-(tetrahydro-2H-pyran-2-yloxy)ethoxy-d4)ethyl)piperazin-1-yl)dibenzo[b,f][1,4]thiazepine 8c. (E)-11-(4-(2-(2-(tetrahydro-2H-pyran-2-yloxy)ethoxy-d4)ethyl)piperazin-1-yl)dibenzo[b,f][1,4]thiazepine,

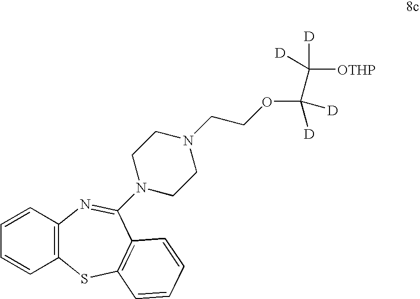

was prepared as described above. This material was purified via column chromatography (0%-5% MeOH/CH₂Cl₂) to afford 28% yield.

¹H NMR (CDCl₃) δ 1.50-1.82 (m, 6H), 2.50-2.71 (m, 4H), 2.64 (t, 2H, J=5.5), 3.46-3.61 (m, 5H), 3.66 (t, 2H, J=5.8), 3.83-3.88 (m, 1H), 4.62 (t, 1H, J=3.9), 6.86 (td, 1H, J=1.5, 7.9), 7.06 (dd, 1H, J=1.5, 7.9), 7.15 (td, 1H, J=1.5, 7.9), 7.25-7.33 (m, 3H), 7.37 (dd, 1H, J=1.5, 7.9), 7.48-7.51 (m, 1H). LCMS m/z 472.0 [M+H].

Preparation of Compounds of Formula I from 8. A round-bottom flask was charged with 8 (0.416 mmol), toluene (1.3 mL), water (0.9 mL), and concentrated HCl (0.141 mL). The biphasic mixture was stirred vigorously at RT for 2 h-3 h. The reaction mixture was diluted with toluene (5 mL) and water (5 mL) and transferred to a separatory funnel. The layers were shaken and separated, and the aqueous layer was washed with toluene (5 mL). The organic layers were discarded and the aqueous layer was diluted with fresh toluene (10 mL). A solution of 10% aq K₂CO₃ was added slowly until the pH of the aqueous layer reached approximately 10. The cloudy biphasic mixture was transferred to a separatory funnel and the layers were shaken and separated. The toluene layer was reserved, and the aqueous layer was extracted with toluene (2×5 mL). The organic layers were combined, washed with brine, dried over magnesium sulfate, filtered and concentrated on a rotary evaporator to afford a compound of Formula I.

(E)-2-(2-(4-(dibenzo[b,f][1,4]thiazepin-11-yl)piperazin-1-yl)ethoxy-d4)ethanol-Compound 100.

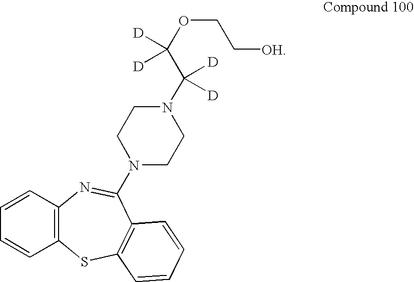

81% yield.

¹H NMR (CDCl₃) δ 2.51-2.58 (m, 2H), 2.62-2.71 (m, 2H), 3.38-3.75 (m, 4H), 3.61-3.63 (m, 2H), 3.69-3.71 (m, 2H), 6.88 (td, 1H, J=1.5, 7.6), 7.06 (dd, 1H, J=1.5, 7.9), 7.14-7.19 (m, 1H), 7.28-7.35 (m, 3H), 7.38 (dd, 1H, J=1.5, 7.6), 7.50 (dt, 1H, J=1.5, 7.9). LCMS m/z 388.0 [M+H].

(E)-2-(2-(4-(dibenzo[b,f][1,4]thiazepin-11-yl)piperazin-1-yl)ethoxy-d4)ethanol-d4-Compound 101.

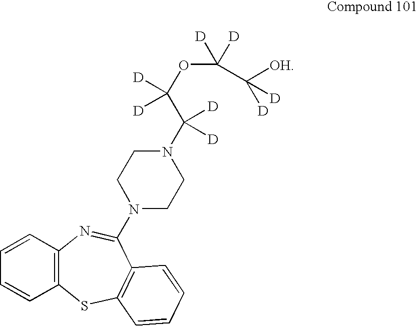

84% yield.

¹H NMR (CDCl₃) δ 2.52-2.58 (m, 2H), 2.63-2.71 (m, 2H), 3.45-3.79 (m, 4H), 6.88 (td, 1H, J=1.5, 7.6), 7.07 (dd, 1H, J=1.5, 7.9), 7.16 (td, 1H, J=1.5, 7.6), 7.27-7.34 (m, 3H), 7.39 (dd, 1H, J=1.5, 7.6), 7.50 (d, 1H, J=7.3). LCMS m/z 392.0 [M+H].

(E)-2-(2-(4-(dibenzo[b,f][1,4]thiazepin-11-yl)piperazin-1-yl)ethoxy)ethanol-d4-Compound 102.

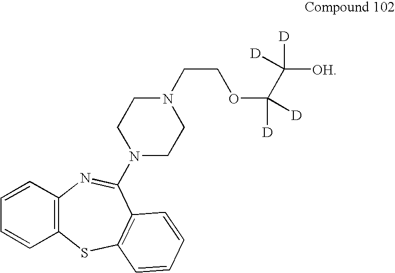

89% yield.

¹H NMR (CDCl₃) δ 2.53-2.58 (m, 2H), 2.63 (t, 2H, J=5.4), 2.60-2.71 (m, 2H), 3.38-3.76 (m, 4H), 3.68 (t, 2H, J=5.4), 6.88 (td, 1H, J1.5, 7.6), 7.07 (dd, 1H, J=1.5, 7.9), 7.14-7.19 (m, 1H), 7.28-7.36 (m, 3H), 7.38 (dd, 1H, J=1.5, 7.6), 7.51 (dt, 1H, J=1.5, 7.6). LCMS m/z 388.0 [M+H].

### Example 2

### Antagonism of Apomorphine-Induced Hyperactive in Rats

This test has been described by Swerdlow and Koob [Pharmacol Biochem Behav, 23: 303 (1985)]. Rats that are administered amphetamine at a moderate dose become hyperactive. The hyperactivity can last for several hours, and can be measured in various ways, for example, by counting the number of times the rat walks from one end of a long alley to the other end. The physiological basis for amphetamine-induced hyperactivity is thought to be the release of excessive amounts of dopamine in the brain. The hyperactivity of anphetamine-treated rats can be antagonized (prevented) by pretreatment with dopamine-blocking agents. The antagonism of amphetamine-induced hyperactivity in rats is, therefore, an indication of the potential dopamine-blocking and potential antipsychotic activity of the agent. A compound of the present invention as the HCl salt or the vehicle is administered orally to 20 rats and amphetamine was then injected intraperitoneally. Activity (walking back and forth in a long alley) is recorded for two hours.

### Example 3

### Affect of Test Compound on Rat Striatal Levels of Dihydroxyphenylacetic Acid (DOPAC) and Homovanillic Acid (HVA)

Among the various pharmacological effects of antipsychotics, their action as dopamine antagonists in the brain has been extensively investigated. Enhancement of dopamine metabolism (dihydroxyphenylacetic acid and homovanillic acid (DOPAC and HVA)) by antipsychotic agents has been attributed to a blockade of dopamine receptors [A. Carlson and M. Lindquist, Acta Pharmac Tox, 1963, 20: 140]. The effects of a compound of the invention on DOPAC and HVA levels in the rat striatum are measured by HPLC using electrochemical detection according to the method of Saller and Salama [J Chromatogr 1984, 309: 287]. A compound of the invention (HCl salt) is suspended in a vehicle and administered intraperitoneally (i.p.) to eight Sprague Dawley rats. Results are recorded.

### Example 4

### Conditioned Avoidance in Squirrel Monkeys

The conditioned avoidance test has been described by Herz, A., Int Rev Neurobiol 1960, 2: 229-277. In this test, a warning stimulus is presented for five seconds. The monkeys are trained to press a lever to turn off the warning stimulus thereby avoiding the delivery of electric shocks at 1/sec for 10 seconds that would begin at the end of the warning stimulus. If there is no response during the warning stimulus (no avoidance response) and the shocks begin, a response during the shocks stops the shocks. Trials of this type are repeated every minute for six hours. Antipsychotic drugs produce a marked reduction in responding to the warning stimulus. A compound of the present invention HCl salt is administered orally and the conditioned avoidance test is administered. The results are recorded.

### Example 5

### Test for Production of Acute Dystonia, Acute Dyskinesia, and Tardive Dyskinesia

One test for predicting whether or not a potential antipsychotic drug will produce involuntary movements of the type described in this application, such as acute dystonia and acute dyskinesia, is in the haloperidol-sensitized and drug-naive cebus monkey. Such tests are described by Barany, Haggstrom and Gunne, Acta Pharmacol et Toxicol 1983, 52:86; J. Liebman and R. Neale, Psychopharm 1980, 68:25-29; and B. Weiss and S. Santelli, Science 1978, 200:799-801. (Also see a discussion of test results in A. Gunne and S. Barany, Psychopharmacol 1979, 63:195-198). Also, antipsychotic drugs that are known to produce tardive dyskinesia in schizophrenic patients produce acute dyskinetic and dystonic reactions in the haloperidol-sensitized cebus monkey. Clozapine, the only antipsychotic drug for which there has been no tardive dyskinesia reported, does not produce a dyskinetic reaction in sensitized cebus monkeys. A compound of the invention, clozapine, thioridazine or haloperidol is each orally administered to sensitized cebus monkeys. They are then observed in their home cages continuously for eight hours and occurrences of dyskinetic reactions noted.

### Example 6

### Evaluation of Metabolic Stability

Certain in vitro liver metabolism studies have been described previously in the following references, each of which is incorporated herein in their entirety: Obach, R S, Drug Metab Disp, 1999, 27:1350; Houston, J B et al., Drug Metab Rev, 1997, 29:891; Houston, J B, Biochem Pharmacol, 1994, 47:1469; Iwatsubo, T et al., Pharmacol Ther, 1997, 73:147; and Lave, T, et al., Pharm Res, 1997, 14:152.

Microsomal Assay: The metabolic stability of compounds of Formula I is tested using pooled liver microsomal incubations. Full scan LC-MS analysis is then performed to detect major metabolites. Samples of the test compounds, exposed to pooled human liver microsomes, are analyzed using HPLC-MS (or MS/MS) detection. For determining metabolic stability, multiple reaction monitoring (MRM) is used to measure the disappearance of the test compounds. For metabolite detection, Q1 full scans are used as survey scans to detect the major metabolites.

Experimental Procedures: Human liver microsomes are obtained from a commercial source (e.g., Absorption Systems L.P. (Exton, Pa.)). The incubation mixtures are prepared as follows:

Reaction Mixture Composition

| | | |
|---|---|---|
| Liver Microsomes | 1.0 | mg/mL |
| NADPH | 1 | mM |
| Potassium Phosphate, pH 7.4 | 100 | mM |
| Magnesium Chloride | 10 | mM |
| Test Compound | 1 | μM. |

Incubation of Test Compounds with Liver Microsomes: The reaction mixture, minus cofactors, is prepared. An aliquot of the reaction mixture (without cofactors) is incubated in a shaking water bath at 37° C. for 3 minutes. Another aliquot of the reaction mixture is prepared as the negative control. The test compound is added into both the reaction mixture and the negative control at a final concentration of 1 μM. An aliquot of the reaction mixture is prepared as a blank control, by the addition of plain organic solvent (not the test compound). The reaction is initiated by the addition of cofactors (not into the negative controls), and then incubated in a shaking water bath at 37° C. Aliquots (200 μL) are withdrawn in triplicate at multiple time points (e.g., 0, 15, 30, 60, and 120 minutes) and combined with 800 μL of ice-cold 50/50 acetonitrile/dH₂O to terminate the reaction. The positive controls, testosterone and propranolol, as well as Compound 1, are each run simultaneously with the test compounds in separate reactions.

All samples are analyzed using LC-MS (or MS/MS). An LC-MRM-MS/MS method is used for metabolic stability. Also, Q1 full scan LC-MS methods are performed on the blank matrix and the test compound incubation samples. The Q1 scans serve as survey scans to identify any sample unique peaks that might represent the possible metabolites. The masses of these potential metabolites can be determined from the Q1 scans.

SUPERSOMES™ Assay. Various human cytochrome P450-specific SUPERSOMES™ are purchased from Gentest (Woburn, Mass., USA). A 1.0 mL reaction mixture containing 25 pmole of SUPERSOMES™, 2.0 mM NADPH, 3.0 mM MgCl, and 1 μM of a compound of Formula I or II in 100 mM potassium phosphate buffer (pH 7.4) was incubated at 37° C. in triplicate. Positive controls contain 1 μM of Compound 1 instead of a compound of formula I. Negative controls used Control Insect Cell Cytosol (insect cell microsomes that lacked any human metabolic enzyme) purchased from GenTest (Woburn, Mass., USA). Aliquots (50 μL) are removed from each sample and placed in wells of a multi-well plate at various time points (e.g., 0, 2, 5, 7, 12, 20, and 30 minutes) and to each aliquot is added 50 μL of ice cold acetonitrile with 3 μM haloperidol as an internal standard to stop the reaction.

Plates containing the removed aliquots are placed in −20° C. freezer for 15 minutes to cool. After cooling, 100 μL of deionized water is added to all wells in the plate. Plates are then spun in the centrifuge for 10 minutes at 3000 rpm. A portion of the supernatant (100 μL) is then removed, placed in a new plate and analyzed using Mass Spectrometry.

Without further description, it is believed that one of ordinary skill in the art can, using the preceding description and the illustrative examples, make and utilize the compounds of the present invention and practice the claimed methods. It should be understood that the foregoing discussion and examples merely present a detailed description of certain preferred embodiments. It will be apparent to those of ordinary skill in the art that various modifications and equivalents can be made without departing from the spirit and scope of the invention. All the patents, journal articles and other documents discussed or cited above are herein incorporated by reference.

1. A compound of formula I: 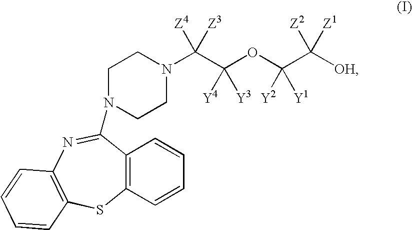
or a salt, hydrate, or solvate thereof, wherein:
each Z is independently selected from hydrogen or deuterium;
each Y is independently selected from hydrogen, deuterium or fluorine; and
at least one Z is deuterium.

2. The compound of claim 1, wherein Y¹ and Y² are each independently deuterium or fluorine.

3. The compound of claim 2, wherein Y¹ and Y² are each deuterium

4. The compound of claim 1, wherein Y³ and Y⁴ are each independently deuterium or fluorine.

5. The compound of claim 4, wherein Y³ and Y⁴ are each deuterium.

6. The compound of claim 1, wherein Z¹ and Z² are each deuterium.

7. The compound of claim 1, wherein Z³ and Z⁴ are each deuterium.

8. The compound of claim 1, selected from:

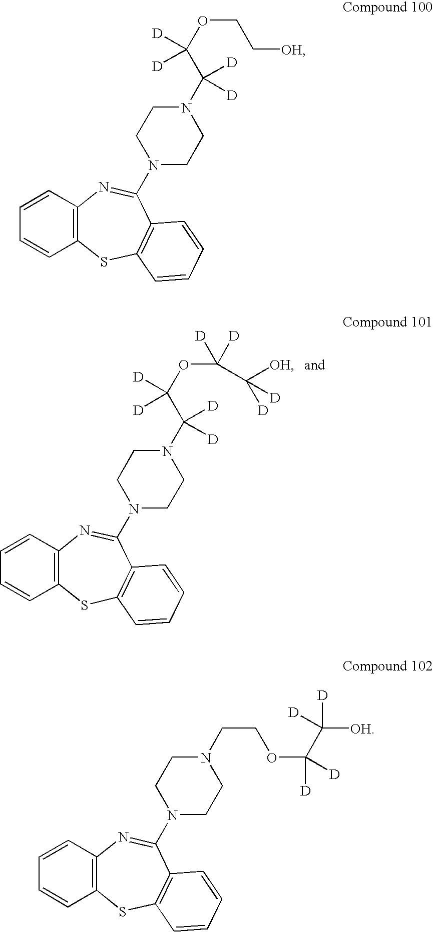

9. The compound of claim 1, wherein any atom not designated as deuterium is present at its natural isotopic abundance.

10. A pyrogen-free composition comprising a compound of claim 1; and an acceptable carrier.

11. The composition of claim 10 formulated for pharmaceutical administration, wherein the carrier is a pharmaceutically acceptable carrier.

12. The composition of claim 11 further comprising a second therapeutic agent useful in the treatment of a disease or condition selected from schizophrenia; schizoaffective disorders; mania (manic disorder); bipolar I disorder; bipolar II disorder; depression associated with bipolar disorders; unipolar depression; agitation and/or insomnia in Alzheimer's patients; agitation and/or psychosis in dementia and/or Parkinsonism; alcoholism; sleep disorders associated with alcohol abstention in alcoholics; substance-related disorders; generalized agitation; generalized anxiety; anxiety disorders; anxiety neuroses; major depression (major depressive disorder); borderline personality disorder; post-traumatic stress disorder; primary insomnia; dementia; anorexia nervosa; social phobia; manic-depressive psychoses; mood disorders; psychotic disorders; psychosis; and obsessive-compulsive disorder.

13. The composition of claim 12, wherein the second therapeutic agent is selected from sabcomeline; a nicotine acetylcholine alpha 7 receptor agonist; moclobemide; brofaromine; befloxatone; toloxatone; gluoxetine; citalopram; excitalopram; fluvoxamine; sertraline; paroxetine; a dopamine D1 antagonist; zolmitriptan; divalproex; lithium; and guanfacine.

14. The composition of claim 11, wherein the second therapeutic agent is selected from divalproex and lithium.

15. A method of modulating the activity of one or more of: serotonergic 5HT1A or 5HT2 receptors, dopaminergic D1 or D2 receptor, histaminergic H1 receptors, or adrenergic a1 or a2 receptors in a cell comprising contacting the cell with a compound of claim 1.

16. A method of treating a patient suffering from or susceptible to a disease of condition selected from schizophrenia; schizoaffective disorders; mania (manic disorder); bipolar I disorder; bipolar II disorder; depression associated with bipolar disorders; unipolar depression; agitation and/or insomnia in Alzheimer's patients; agitation and/or psychosis in dementia and/or Parkinsonism; alcoholism; sleep disorders associated with alcohol abstention in alcoholics; substance-related disorders; generalized agitation; generalized anxiety; anxiety disorders; anxiety neuroses; major depression (major depressive disorder); borderline personality disorder; post-traumatic stress disorder; primary insomnia; dementia; anorexia nervosa; social phobia; manic-depressive psychoses; mood disorders; psychotic disorders; psychosis; and obsessive-compulsive disorder, comprising the step of administering to the patient mammal in need thereof with a composition of claim 9.

17. The method of claim 16, wherein the patient is suffering from or susceptible to schizophrenia or bipolar I disorder.

18. The method of claim 16, comprising the additional step of co-administering to the patient in need thereof a second therapeutic agent selected from sabcomeline; a nicotine acetylcholine alpha 7 receptor agonist; a serotonin/norepinephrine reuptake inhibitor; moclobemide; brofaromine; befloxatone; toloxatone; gluoxetine; citalopram; excitalopram; fluvoxamine; sertraline; paroxetine; a dopamine D1 antagonist; zolmitriptan; divalproex; lithium; and guanfacine.

19. The method of claim 18, wherein:
a. the patient is suffering from or susceptible to anxiety or anxiety disorder; and the second therapeutic agent is a SSRI or a SNRI.
b. the patient is suffering from or susceptible to Alzheimer's disease or dementia; and the second therapeutic agent is divalproex;
c. the patient is suffering from or susceptible to schizophrenia; and the second therapeutic agent is guanfacine; or
d. the patient is suffering from or susceptible to bipolar I disorder and the second therapeutic agent is selected from lithium and divalproex.
